(19) United States
(12) Patent Application Publication
(10) Pub. No.: US 2018/0344589 A1
(43) Pub. Date: Dec. 6, 2018

(54) AEROSOLIZED DYE AND SYSTEM FOR DELIVERING AEROSOLIZED DYE

(71) Applicant: Jennifer Donya Litowitz, Miami, FL (US)

(72) Inventor: Jennifer Donya Litowitz, Miami, FL (US)

(21) Appl. No.: 16/001,570

(22) Filed: Jun. 6, 2018

Related U.S. Application Data
(60) Provisional application No. 62/515,655, filed on Jun. 6, 2017.

(51) Int. Cl. A61K 8/04; A61Q 5/06; A61Q 5/10

(52) U.S. Cl. CPC A61K 8/046; A61K 2800/4322; A61Q 5/10; A61Q 5/065

(57) ABSTRACT

An aerosolized semi-permanent hair dye is provided for, which is non-toxic to humans and shelf stable, utilizing coal tar dyes, solvents, propellants, neutralizers, colorant treatments and humectants. The invention has the benefit of being easily applied to hair or other keratinous substances and dyes hair for a period longer than currently available spray-in temporary colorants, but shorter than oxidized or otherwise developed permanent hair dyes.

### CLAIM OF PRIORITY

This non-provisional application claims the benefit of U.S. Provisional Patent Application No. 62/515,655 filed on Jun. 6, 2017.

### BACKGROUND OF THE INVENTION

### 1. Field of the Invention

The present invention relates to the field of hair dyes, hair sprays, and the haircare and personal grooming industry. More particularly, the present invention relates to an aerosolized dye for hair or other keratinous fibers utilizing coal tar and system for delivering an aerosolized hair dye.

### 2. Description of the Related Art

While there are multiple hair coloring products presently on the market, these products fall into one of two categories: they are either dyes that are applied to hair or other keratinous fiber mechanically and directly (such as through use of a brush, stick, or other device), or they are coatings that are aerosolized and sprayed onto the hair or other keratinous fiber but do not actually dye (i.e., they sit on top of the hair or other keratinous fiber, providing a layer that creates color instead of altering the color of the hair or other keratinous fiber itself).

The market for hair coloring is large and substantial. In 2016, Business Wire reported that analysts at Technavio forecast that the global hair color market would reach $29.14 billion by 2019, growing at a compound annual growth rate of more than 9%.

Despite this large and growing market, there is no hair dye (or dye for other keratinous fibers) that is aerosolized or otherwise sprayable.

For example, JP2003026553A by Isoda Naoki et al., describes a temporary hair-coloring agent comprising titanated mica, a pigment, an inorganic polymer-based thickening dispersant, and a film-forming agent. This product is not a hair dye, it is a colorized coating for hair.

Colorized coatings for hair are problematic and do not solve the problems solved by the present invention. Colorized coatings for hair are highly temporary as the coloring is provided by a substance that sits on the surface of the hair or other keratinous fiber. Because it sits on the surface of the hair, colorized coatings can be relatively easily removed from the surface of the hair, thus removing the desired coloring of the hair. This also presents other problems as the coating, and thus desired coloring, can be prematurely lost. Further, when the colored coating is removed from the surface of the hair, it can easily run off and contact other surfaces it will stain, such as furniture, upholstery, or clothing.

For example, a consumer may desire to color their hair a bright or ‘unnatural’ color, such as pink, blue, or green, for an event, such as a concert, holiday (such as Halloween), a corporate party, etc., or simply to provide unique highlights or simply try a different color for their hair. Such a consumer is presently faced with two choices: utilize a temporary, ‘coating’ style hair-coloring agent or utilize a permanent hair dye. Neither of these options is desirable. The first option (the temporary hair coating) can easily be accidentally or unintentionally removed from the consumer's hair, such as, for example, if the consumer gets caught in a rain storm; the coloring may also be accidentally transferred to the consumer's clothing, furniture, and other surfaces. None of the foregoing is a desirable outcome. Similarly, the second option (the permanent hair dye) is equally undesirable as it is permanent and therefore requires the consumer to either keep the changed hair color until their hair grows long enough that the color can be cut out or re-dye their hair back to their original color, a process that is time consuming, expensive, and damaging to the hair.

Another example is JPH0987152A to Yuasa Koichi. Koichi describes a dye product that requires a developer, creating an oxidization process to dye a hair. By relying on an oxidizer, Koichi, adds additional, unnecessary steps to the process of dying hair and creates a harsher dying process.

As with the preceding example, Koichi is undesirable because it is more time consuming, harsher, and more permanent.

What is needed is a hair dye that can be aerosolized, thus eliminating the currently messy and laborious process of applying traditional hair dyes, that is not merely a coating, that is safe and non-toxic for human use, that is shelf-stable, and that is semi-permanent, allowing for an easy, mess-free application that will not be prematurely removed from the user's hair.

### SUMMARY OF THE INVENTION

The present invention relates to an aerosolized dye, and system for delivering an aerosolized dye, for use on hair, fur, or other keratinous fibers.

The present invention uses wet coal tar as a dye/colorant.

The present invention utilizes a specific pH range of between 6 and 9 to produce the dye effect and thus eliminates the need to use an oxidizer or intermediates such as peroxide.

The present invention utilizes a particle size between 10 and 40 microns.

One of the benefits of the present invention is that it allows for an aerosolized hair dye that is easier and cleaner to apply than current methods.

Another benefit of the present invention is it is a dye instead of a coating and is thus able to withstand multiple washings without color degradation. A further benefit is that, unlike a coating, the present invention will not prematurely be removed from a user's hair resulting in undesired transfer onto other surfaces, such as upholstery, clothing, or bedding.

Yet another benefit of the present invention is that the particle size results in the spray being non-toxic to humans as the particle size is too large to permit aspiration or inhalation by a human. A further benefit of the present invention is that the particle size helps prevent overspray and transfer of the dye onto surfaces where dye is not desired, such as clothing or floors.

Still a further benefit of the present invention is, by not using an oxidizer or developing agent, the present invention is simpler to use, possesses enhanced shelf-stability and longevity, and can be washed out after approximately five washes, allowing users to try different colors, such as unnatural colors like blue, violet, or pink.

Further elements and benefits of the invention are described in the attachments hereto.

### BRIEF DESCRIPTION OF THE DRAWINGS AND EXAMPLE FORMULAE

The accompanying drawings and example formulae are attached hereto, are incorporated in and constitute part of this specification, and illustrate embodiments of the invention, and together with the description, serve to explain the principles of the invention. The embodiments illustrated herein are presently preferred, it being understood, however, that the invention is not limited to the precise arrangements and instrumentalities shown.

FIG. 1 is a chart showing average particle size distribution for one test formulation of the claimed invention.

FIG. 2 is a chart showing average particle size distribution for another test formulation of the claimed invention.

FIG. 3 is a chart showing average particle size distribution for yet another test formulation of the claimed invention.

FIG. 4 is a table showing the stability of one test formulation of the claimed invention.

FIG. 5 is a table showing the stability of another test formulation of the claimed invention.

FIG. 6 is a table showing the stability of yet another test formulation of the claimed invention.

### DETAILED DESCRIPTION OF THE INVENTION

The present invention relates to an aerosolized spray for the semi-permanent dying of hair or other keratinous fibers.

With respect to all formulations of the invention (examples of which are described hereinbelow), the basic compounding of the invention is the following: (1) charge solvent; (2) charge film former as necessary to prepare the proper pH required for the specific color being used; (3) add neutralizer for film former to create the proper pH range for the specific color being used; (4) adding a plasticizer; (5) adding any fragrances desired (which are optional); (6) adding any extracts (which are optional); (7) adding any other additional compounds or materials which may be desired to provide additional features to the user (such as anti-dandruff agents); (8) engaging a premix procedure comprising (a) charging appropriate volume by weight of water and then adding the specific coal tar dye or dyes being used to achieve the desired color result; (b) mixing until no dye powder remains upon visual inspection; and (c) adding the premix to the main mix tank and mixing until a homogenous solution is formed; and (9) adding any shimmer ingredients desired (such as Covapearls from Sensient).

As noted, different colors require a different specific pH for use. However, in all instances of use and testing for wet coal tar dye, the pH range is between 6.0 and 9.0, with some specific color dies requiring narrower ranges for optimal performance, such as a pH range between 6.0 to 7.0.

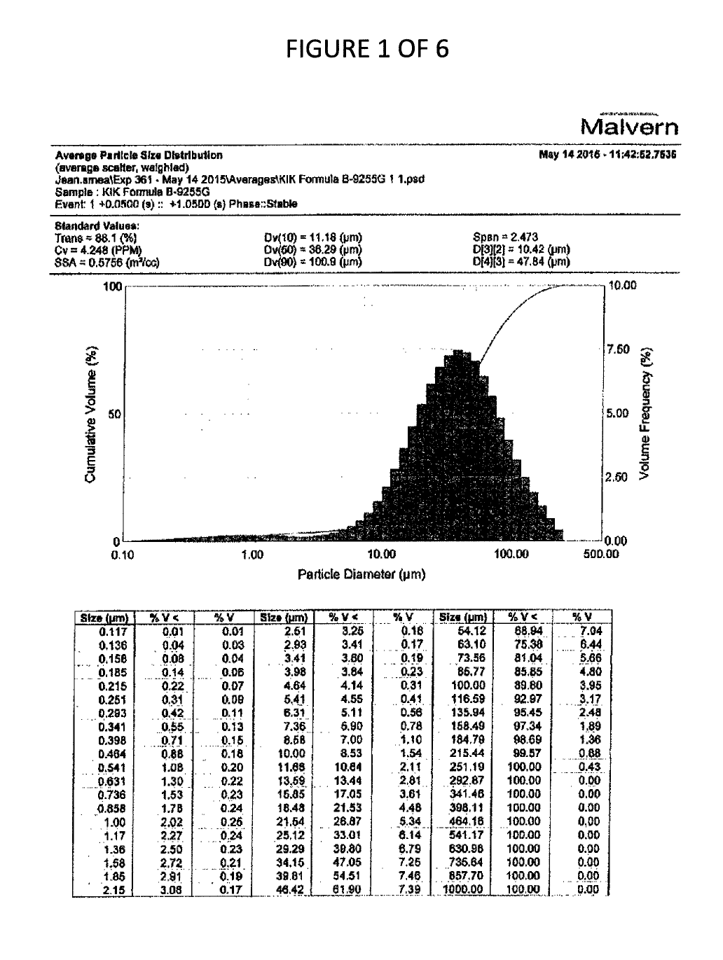

Due to the requirement for slightly different pH depending on the desired color, the individual chemical formulations for the different colors will also need to vary. However, in all instances, the formulation must result in a pH that allows for the particular color to be aerosolized, for the aerosolized spray to be comprised of particles with specific diameters to enable ease of application while remaining non-toxic to humans, as shown in FIGS. 1, 2, and 3 hereto, and for the resulting spray to be a semi-permanent. Examples of specific formulations are shown in Examples 1 through 5 herein.

However, all formulations utilize the same basic ingredients: a solvent or solvents, a propellant or propellants, a neutralizer or neutralizers, a colorant treatment or colorant treatments, a colorant or colorants, and a humectant or humectants.

The solvent or solvents used are ideally denatured alcohol (which may include t-Butyl Alcohol and/or Denatonium Benzoate), water (aqua/eau), distilled water (aqua/eau), and/or Cyclopentasiloxane.

The propellants used are ideally Hydrofluorocarbon 152a and/or Isobutane.

The neutralizer used is ideally Aminomethyl Propanol.

The colorant treatment or treatments are ideally Triethoxycarylylsilane and/or Synthetic Fluorphlogopite.

The colorants used are coal tar dies or similar, such as, ideally, Cl 77891 (Titanium Dioxide), Mica, Basic Red 51, Basic Blue 99, HC Blue No. 2, HC Orange No. 1, HC Yellow #4, Disperse Blue 377, HC Yellow No. 2, 4-Amino-Nitrophenol, 3-Nitro-p-Hydroxyethylaminophenol, HC Red No. 15, Disperse Black 9

The humectant used is ideally Glycerine.

In some formulations, multiple colorants may be required in order to obtain the desired shade or hue.

Optionally, polymers may be included, such as Phenyl Trimethicone and/or PEG-12 Dimethicone (silicone polymer).

Optionally, a plasticizer may be included, such as Triethyl Citrate.

Optionally, additional chemicals and/or compounds designed to appeal to consumers (“Marketing Compounds”), which further enhance the invention, may be included, such as Hydrolyzed Quinoa Protein, Helianthus annuus (Sunflower) Seed Extract, Persea gratissima (Avocado) Fruit Extract, Calendula officinalis Flower Extract, Aloe barbadensis Leaf Extract, Camellia sinensis Leaf Extract, Vitis vinifera (Grape) Seed Extract, and/or Dimethyl Stearamine.

Additionally, if optional additional chemicals and/or compounds designed to appeal to consumers are utilized, preservatives may also need to be included to protect those additional chemicals and compounds. These preservatives may include PPG-26-Buteth-26, PEG-40 Hydrogenated Castor Oil, HC Blue No. 15, Panthenol, Ethylhexyl Methoxycinnamate, and/or Basic Yellow 87.

Optionally, additional preservatives may be used to maintain shelf stability and longevity of the invention.

Optionally, perfume or other fragrances may be added, such as Benzyl Benzoate, Butylphenyl Methylpropional, Hexyl Cinnamaldehyde, Hydroxyisohexyl 3-Cyclohexene 3 and carboxaldehyde, Limonene, and/or Linalool.

Optionally a pH adjuster or adjustors, such as Aminomethyl Propanol, may be utilized in order to adjust the pH value of the overall invention to the pH necessary to obtain proper functionality of the invention as set forth in this specification.

Optionally a conditioning agent or agents, such as Phenyl Trimethicone, may be included to improve useability and desirability of the invention to the consuming public.

Optionally, other chemicals and/or compounds that add desirable other features (such as anti-dandruff) may be included.

Other excipients may also be included.

An additional benefit of the claimed invention is that, utilizing the methods and formulations disclosed and described herein, the product is non-toxic to humans.

A further understanding of the invention may be obtained from specific formulations set forth in the following five examples.

### Example 1

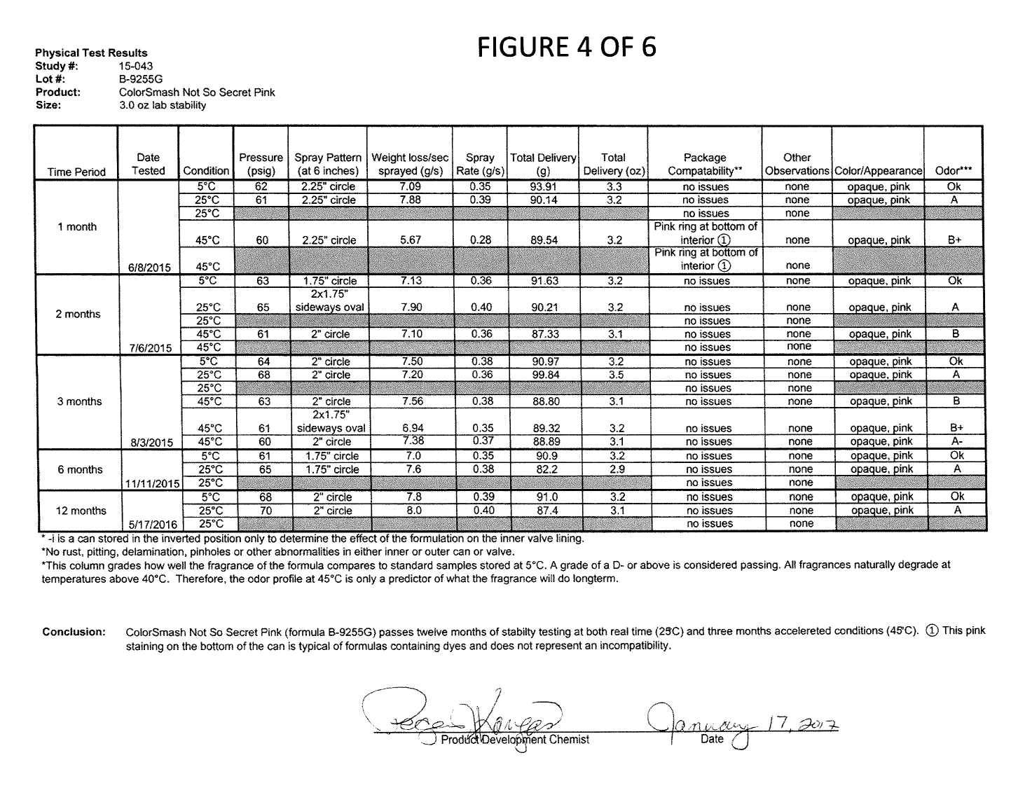

The following example shows the maximum allowable percentage by weight, and ideal percentage by weight, of each chemical and compound comprising the ‘pink’ colored embodiment of the invention, as shown in FIGS. 1 and 4:

| | INCI/Chemical | Maximum | Breakdown |
|---|---|---|---|
| | Names | allowable | by |
| | present in | percentage | Percentage |
| | the raw | by | wt. for each |
| Trade Name | material | weight | substance |
| Tulstar 152a | Hydrofluorocarbon 152a | 40.114 | 40.114 |
| SD Alcohol 40B 200 proof | Alcohol Denat. | 46.290146 | 46.290146 |
| AeroN A31 | Isobutane | 5.886 | 5.886 |
| Amphomer LV71 | Octylacrylamide/ | 4.32 | 4.32 |
| | Acrylates/ | | |
| | Butylaminoethyl | | |
| | Methacrylate | | |
| | Copolymer | | |
| Water | Water (Aqua/Eau) | 1.62265 | 1.62265 |
| Ultra PC 1000 | Aminomethyl Propanol | 0.8316 | 0.8316 |
| Covapearl Pink 433 AQ | Mica | 0.378 | 0.20034 |
| Covapearl Pink 433 AQ | PEG-12 Dimethicone | 0.378 | 0.00756 |
| Covapearl Pink 433 AQ | Titanium Dioxide | 0.378 | 0.1701 |
| | (Cl77891) | | |
| Sensient Arianor | Basic Red 51 | 0.0648 | 0.0648 |
| Cherry Red 306008 | | | |
| Dow Corning Fluid 245 | Cyclopentasiloxane | 0.216 | 0.216 |
| Citroflex 2 | Triethyl Citrate | 0.054 | 0.054 |
| Dow corning 556 fluid | Phenyl Trimethicone | 0.054 | 0.054 |
| Enchanted Blossom | Fragrance | 0.165 | 0.14838 |
| Enchanted Blossom | Linalool | 0.165 | 0.00396 |
| Enchanted Blossom | Benzyl Benzoate | 0.165 | 0.00304 |
| Enchanted Blossom | Limonene | 0.165 | 0.003 |
| Enchanted Blossom | Butylphenyl | 0.165 | 0.00297 |
| | Methylpropional | | |
| Enchanted Blossom | Hydroxyisohexyl | 0.165 | 0.00178 |
| | 3-Cyclohexene | | |
| | Carboxaldehyde | | |
| Enchanted Blossom | Hexyl Cinnamal | 0.165 | 0.00158 |
| | Hydrolyzed | 0.0016725 | 0.00129 |
| | Quinoa Protein | | |
| | Phenoxyethanol | 0.0016725 | 0.00003 |
| | Methylparaben | 0.0016725 | 0.00001 |
| | Ethylparaben | 0.0016725 | 0.00001 |
| | Disodium EDTA | 0.0016725 | 0.00001 |
| Extract blend M6941WS | Citric acid | 0.002745 | 0.000013 |
| Extract blend M6941WS | Sodium Benzoate | 0.002745 | 0.000013 |
| Extract blend M6941WS | Potassium Sorbate | 0.002745 | 0.000013 |
| Extract blend M6941WS | Helianthus Annuus | 0.002745 | 0.000009 |
| | (Sunflower) Seed Extract | | |
| Extract blend M6941WS | Persea Gratissima | 0.002745 | 0.000009 |
| | (Avocado) Fruit Extract | | |
| Extract blend M6941WS | Calendula Officinalis | 0.002745 | 0.000009 |
| | Flower Extract | | |
| Extract blend M6941WS | Aloe Barbadensis | 0.002745 | 0.000009 |
| | Leaf Extract | | |
| Extract blend M6941WS | Camllia Sinensis | 0.002745 | 0.000009 |
| | Leaf Extract | | |
| Extract blend M6941WS | Vitis Vinifera (Grape) | 0.002745 | 0.000009 |
| | Seed Extract | | |
| Glycerin | Glycerin | 0.002745 | 0.002651 |

### Example 2

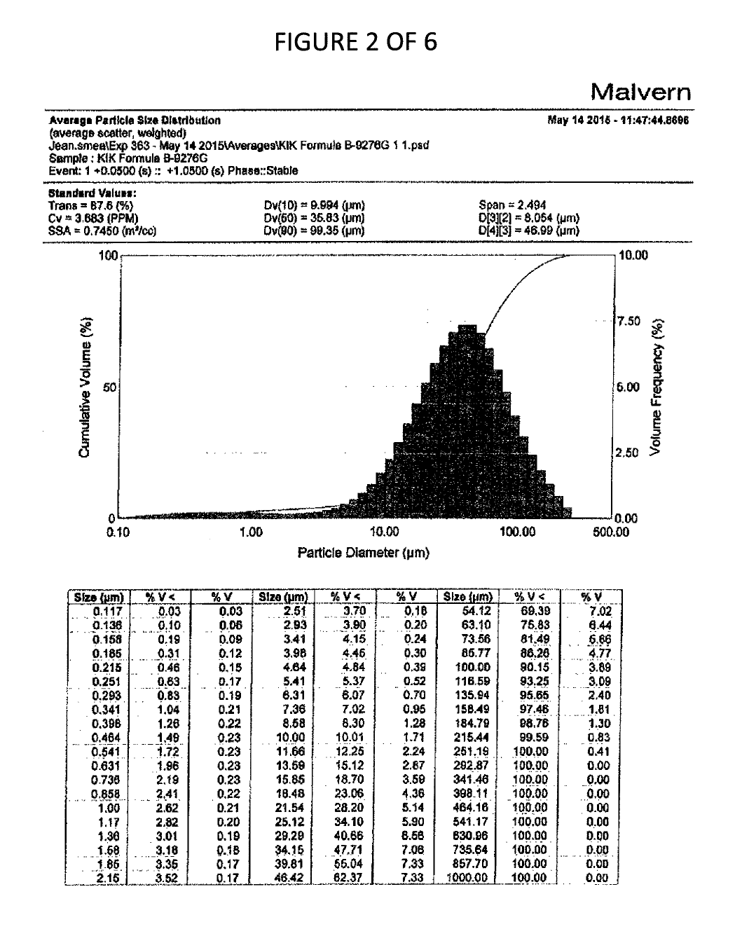

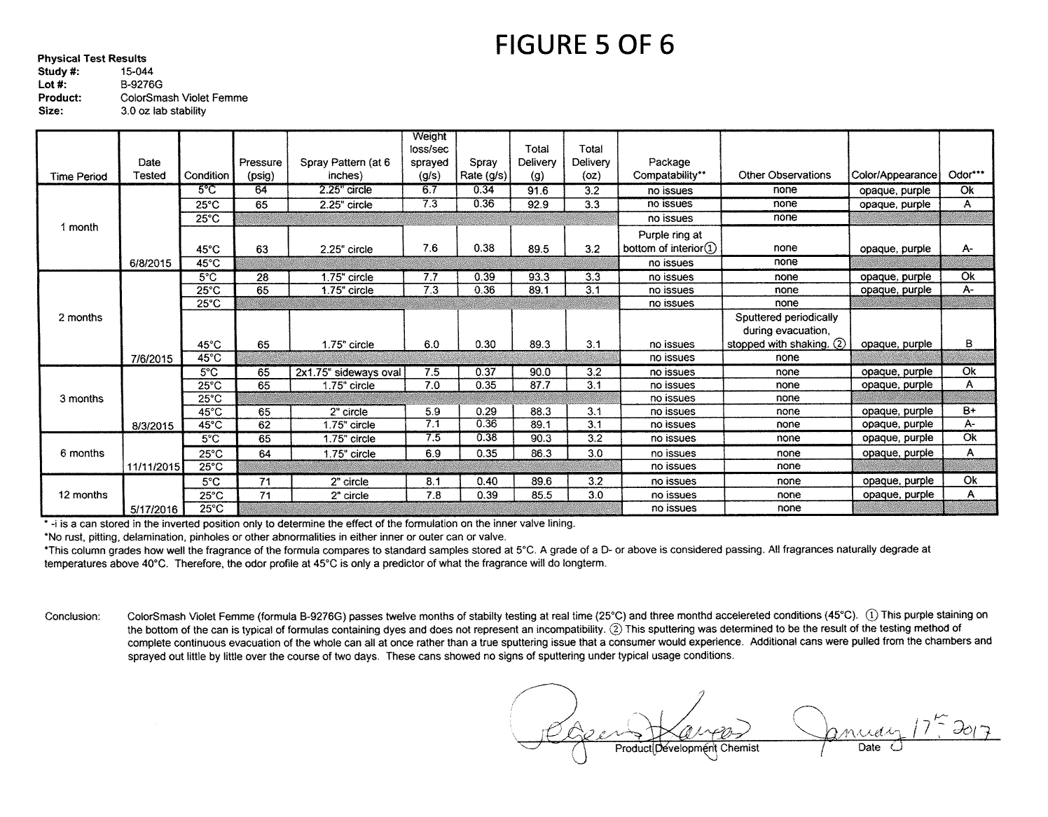

The following example shows the maximum allowable percentage by weight, and ideal percentage by weight, of each chemical and compound comprising the ‘purple’ (a/k/a ‘Violet Femme’) colored embodiment of the invention, as shown in FIGS. 2 and 5:

| | | | Breakdown |
|---|---|---|---|
| | INCI/ | Maximum | by |
| | Chemical Names | allowable | Percentage |
| | present in | percentage | wt. |
| | the raw | by | for each |
| Trade Name | material | weight | substance |
| SD Alcohol 40B | Alcohol Denat. | 46.129746 | 46.129746 |
| 200 proof | | | |
| Tulstar 152a | Hydrofluorocarbon 152a | 40.114 | 40.114 |
| AeroN A31 | Isobutane | 5.886 | 5.886 |
| Amphomer LV71 | Octylacrylamide/ | 4.32 | 4.32 |
| | Acrylates/ | | |
| | Butylaminoethyl | | |
| | Methacrylate Copolymer | | |
| Water | Water (Aqua) | 1.62265 | 1.62265 |
| Ultra PC 1000 | Aminomethyl Propanol | 0.8316 | 0.8316 |
| Dow Corning Fluid 245 | Cyclopentasiloxane | 0.216 | 0.216 |
| Covapearl Mini | Titanium Dioxide | 0.4888 | 0.273728 |
| Star Purple 5331 | (Cl77891) | | |
| Covapearl Mini | Synthetic | 0.4888 | 0.215072 |
| Star Purple 5331 | Fluorphlogopite | | |
| Citroflex 2 | Triethyl Citrate | 0.054 | 0.054 |
| Dow corning 556 fluid | Phenyl Trimethicone | 0.054 | 0.054 |
| Sensient Arianor | Basic Blue 99 | 0.1144 | 0.0572 |
| Tyrian Purple 306021 | | | |
| Sensient Arianor | Basic Red 51 | 0.1144 | 0.0572 |
| Tyrian Purple 306021 | | | |
| Enchanted Blossom | Fragrance | 0.165 | 0.14838 |
| Enchanted Blossom | Linalool | 0.165 | 0.00396 |
| Enchanted Blossom | Benzyl Benzoate | 0.165 | 0.00304 |
| Enchanted Blossom | Limonene | 0.165 | 0.003 |
| Enchanted Blossom | Butylphenyl | 0.165 | 0.00297 |
| | Methylpropional | | |
| Enchanted Blossom | Hydroxyisohexyl | 0.165 | 0.00178 |
| | 3-Cyclohexene | | |
| | Carboxaldehyde | | |
| Enchanted Blossom | Hexyl Cinnamal | 0.165 | 0.00158 |
| Quinoa extract | Hydrolyzed | 0.00135 | 0.00129 |
| VP9760000W | Quinoa Protein | | |
| Quinoa extract | Phenoxyethanol | 0.00135 | 0.00003 |
| VP9760000W | | | |
| Quinoa extract | Methylparaben | 0.00135 | 0.00001 |
| VP9760000W | | | |
| Quinoa extract | Ethylparaben | 0.00135 | 0.00001 |
| VP9760000W | | | |
| Quinoa extract | Disodium EDTA | 0.00135 | 0.00001 |
| VP9760000W | | | |
| Extract blend M6941WS | Potassium Sorbate | 0.002745 | 0.000013 |
| Extract blend M6941WS | Citric acid | 0.002745 | 0.000013 |
| Extract blend M6941WS | Sodium Benzoate | 0.002745 | 0.000013 |
| Extract blend M6941WS | Helianthus Annuus | 0.002745 | 0.000009 |
| | (Sunflower) Seed Extract | | |
| Extract blend M6941WS | Persea Gratissima | 0.002745 | 0.000009 |
| | (Avocado) Fruit Extract | | |
| Extract blend M6941WS | Calendula Officinalis | 0.002745 | 0.000009 |
| | Flower Extract | | |
| Extract blend M6941WS | Aloe Barbadensis | 0.002745 | 0.000009 |
| | Leaf Extract | | |
| Extract blend M6941WS | Camllia Sinensis | 0.002745 | 0.000009 |
| | Leaf Extract | | |
| Extract blend M6941WS | Vitis Vinifera (Grape) | 0.002745 | 0.000009 |
| | Seed Extract | | |
| Glycerin | Glycerin | 0.002745 | 0.002651 |

### Example 3

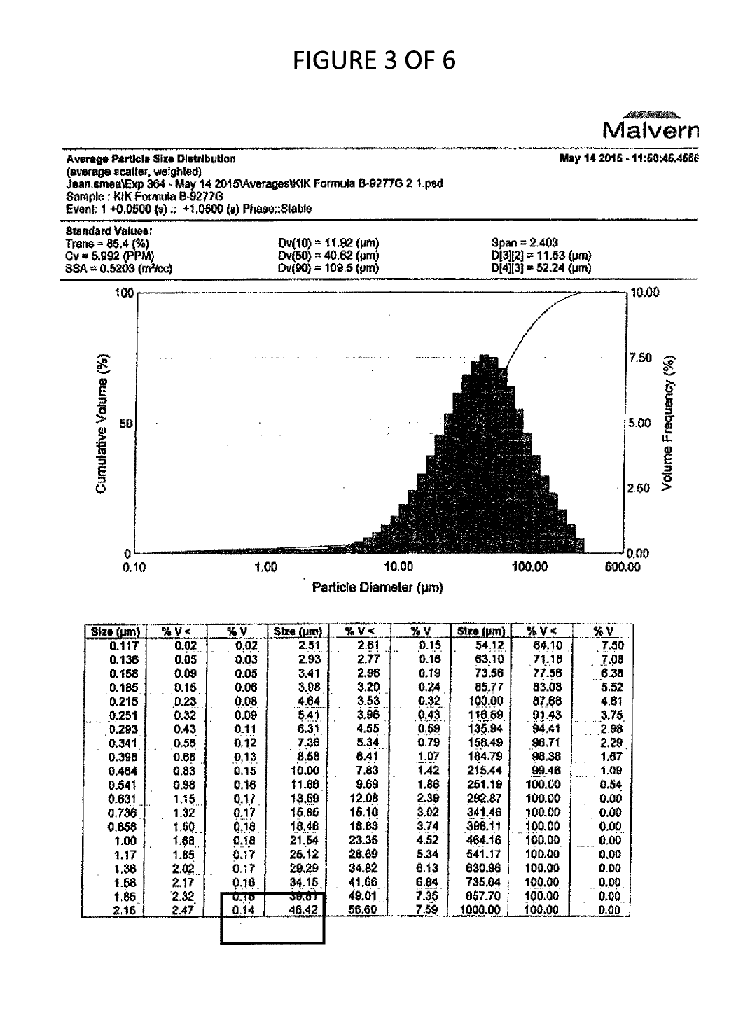

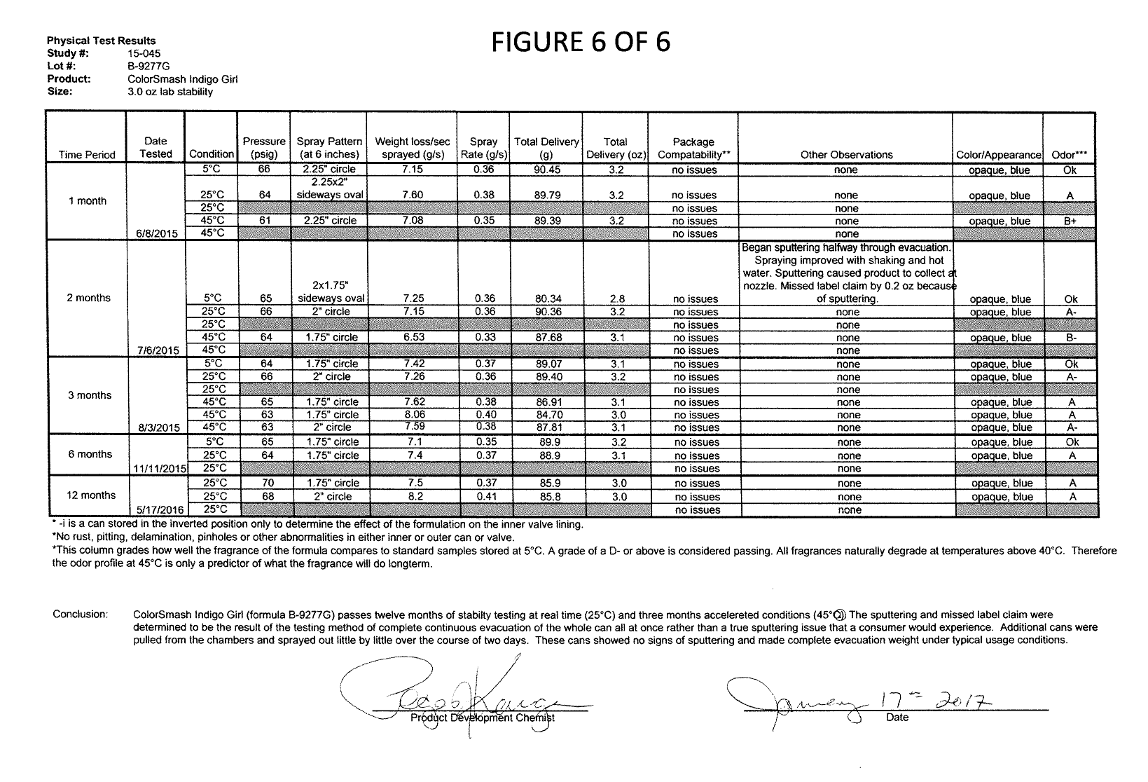

The following example shows the maximum allowable percentage by weight, and ideal percentage by weight, of each chemical and compound comprising the ‘indigo’ (a/k/a ‘Indigo Girl’) colored embodiment of the invention, as shown in FIGS. 3 and 6:

| | INCI/Chemical | Maximum | Breakdown by |
|---|---|---|---|
| | Names present | allowable | Percentage |
| | in the raw | percentage | wt. for each |
| Trade Name | material | by weight | substance |
| SD Alcohol 40B 200 proof | Alcohol Denat. | 45.754146 | 45.75415 |
| Tulstar 152a | Hydrofluorocarbon 152a | 40.11 | 40.11000 |
| AeroN A31 | Isobutane | 5.886 | 5.88600 |
| Amphomer LV71 | Octylacrylamide/ | 4.32 | 4.32000 |
| | Acrylates/ | | |
| | Butylaminoethyl | | |
| | Methacrylate Copolymer | | |
| Water | Water (Aqua/Eau) | 1.62265 | 1.62265 |
| Ultra PC 1000 | Aminomethyl Propanol | 0.8316 | 0.83160 |
| Dow Corning Fluid 245 | Cyclopentasiloxane | 0.216 | 0.21600 |
| Citroflex 2 | Triethyl Citrate | 0.054 | 0.05400 |
| Dow corning 556 fluid | Phenyl Trimethicone | 0.054 | 0.05400 |
| Sensient Arianor Steel Blue | Basic Blue 99 | 0.1728 | 0.17280 |
| Covapearl Blue 635 AS | Titanium Dioxide | 0.81 | 0.40095 |
| | (Cl77891) | | |
| Covapearl Blue 635 AS | Mica | 0.81 | 0.3888 |
| Covapearl Blue 635 AS | Triethoxycaprylylsilane | 0.81 | 0.02025 |
| Enchanted Blossom | Fragrance | 0.165 | 0.14838 |
| Enchanted Blossom | Linalool | 0.165 | 0.00396 |
| Enchanted Blossom | Benzyl Benzoate | 0.165 | 0.00304 |
| Enchanted Blossom | Limonene | 0.165 | 0.003 |
| Enchanted Blossom | Butylphenyl | 0.165 | 0.00297 |
| | Methylpropional | | |
| Enchanted Blossom | Hydroxyisohexyl | 0.165 | 0.00178 |
| | 3-Cyclohexene | | |
| | Carboxaldehyde | | |
| Enchanted Blossom | Hexyl Cinnamal | 0.165 | 0.00158 |
| Quinoa extract | Hydrolyzed | 0.00135 | 0.00129 |
| VP9760000W | Quinoa Protein | | |
| Quinoa extract | Phenoxyethanol | 0.00135 | 0.00003 |
| VP9760000W | | | |
| Quinoa extract | Methylparaben | 0.00135 | 0.00001 |
| VP9760000W | | | |
| Quinoa extract | Ethylparaben | 0.00135 | 0.00001 |
| VP9760000W | | | |
| Quinoa extract | Disodium EDTA | 0.00135 | 0.00001 |
| VP9760000W | | | |
| Extract blend M6941W5 | Citric acid | 0.002745 | 0.000013 |
| Extract blend M6941W5 | Sodium Benzoate | 0.002745 | 0.000013 |
| Extract blend M6941W5 | Potassium Sorbate | 0.002745 | 0.000013 |
| Extract blend M6941W5 | Helianthus Annuus | 0.002745 | 0.000009 |
| | (Sunflower) Seed Extract | | |
| Extract blend M6941W5 | Persea Gratissima | 0.002745 | 0.000009 |
| | (Avocado) Fruit Extract | | |
| Extract blend M6941W5 | Calendula Officinalis | 0.002745 | 0.000009 |
| | Flower Extract | | |
| Extract blend M6941WS | Aloe Barbadensis | 0.002745 | 0.000009 |
| | Leaf Extract | | |
| Extract blend M6941WS | Camllia Sinensis | 0.002745 | 0.000009 |
| | Leaf Extract | | |
| Extract blend M6941W5 | Vitis Vinifera (Grape) | 0.002745 | 0.000009 |
| | Seed Extract | | |
| Glycerin | Glycerin | 0.002745 | 0.002651 |

### Example 4

The following example shows the maximum allowable percentage by weight, and ideal percentage by weight, of each chemical and compound comprising the ‘natural brown’ colored embodiment of the invention.

| | | Breakdown |
|---|---|---|
| | | by |
| | | Percentage |
| | INCI/Chemical | wt. |
| | Names present in | for each |
| Trade Name | the raw material | substance |
| Specially Denatured Alcohol | Alcohol Denat. | 35.578087 |
| (SDS) 40b-200 Proof | | |
| Specially Denatured Alcohol | t-Butyl Alcohol | 0.042745 |
| (SDS) 40b-200 Proof | | |
| Specially Denatured Alcohol | Denatonium Benzoate | 0.000214 |
| (SDS) 40b-200 Proof | | |
| Amphomer LV-71 Polymer | Octylacrylamide/ | 2.619000 |
| | Acrylates/ | |
| | Butylaminoethyl | |
| | Methacrylate | |
| | Copolymer | |
| Amphomer LV-71 Polymer | Water (Aqua, Eau) | 0.064800 |
| Amphomer LV-71 Polymer | PPG-12 Dimethicone | 0.002700 |
| Amphomer LV-71 Polymer | Benzoic Acid | 0.013500 |
| AMP-ULTRA PC 2000 | Aminomethyl Propanol | 0.513000 |
| AMP-ULTRA PC 2000 | Water (Aqua, Eau) | 0.027000 |
| Dow corning 556 | Phenyl Trimethicone | 0.054000 |
| Comsetic Grade Fluid | | |
| Citroflex 2 | Triethyl Citrate | 0.054000 |
| Volasil 995 | Cyclopentasiloxane | 0.212220 |
| Volasil 995 | Cyclohexasiloxane | 0.002592 |
| Volasil 995 | Cyclotetrasiloxane | 0.001188 |
| CP-AB Extract M6941-WS | Water (Aqua, Eau) | 0.002653 |
| CP-AB Extract M6941-WS | Glycerin | 0.002652 |
| CP-AB Extract M6941-WS | Citric Acid | 0.000014 |
| CP-AB Extract M6941-WS | Potassium Sorbate | 0.000014 |
| CP-AB Extract M6941-WS | Sodium Benzoate | 0.000014 |
| CP-AB Extract M6941-WS | Grapeseed Extract | 0.000009 |
| CP-AB Extract M6941-WS | Helianthus Annuus | 0.000009 |
| | (Sunflower) | |
| | Seed Extract | |
| CP-AB Extract M6941-WS | Persea Gratissima | 0.000009 |
| | (Avocado) Fruit | |
| | Extract | |
| CP-AB Extract M6941-WS | Calendula Officinalis | 0.000009 |
| | Flower Extract | |
| CP-AB Extract M6941-WS | Aloe Barbadensis | 0.000009 |
| | Leaf Extract | |
| CP-AB Extract M6941-WS | Camellia Sinensis | 0.000009 |
| | Leaf Extract | |
| Fragrance: Enchanted | Fragrance (Parfum) | 0.144442 |
| Blossom #5198638 | | |
| Fragrance: Enchanted | Benzyl Benzoate | 0.003528 |
| Blossom #5198638 | | |
| Fragrance: Enchanted | Butylphenyl | 0.003402 |
| Blossom #5198638 | Methylpropional | |
| Fragrance: Enchanted | Hexyl Cinnamaldehyde | 0.001620 |
| Blossom #5198638 | | |
| Fragrance: Enchanted | Hydroxyisohexyl | 0.001620 |
| Blossom #5198638 | 3-Cyclohexene 3 | |
| | and carboxaldehyde | |
| Fragrance: Enchanted | Limonene | 0.003175 |
| Blossom #5198638 | | |
| Fragrance: Enchanted | Linalool | 0.004212 |
| Blossom #5198638 | | |
| Hydrolyzed Quinoa | Water (Aqua, Eua) | 0.000040 |
| Protein Extract | | |
| Hydrolyzed Quinoa | Hydrolyzed | 0.000014 |
| Protein Extract | Quinoa Protein | |
| Hydrolyzed Quinoa | Methylparaben | 0.000000 |
| Protein Extract | | |
| Hydrolyzed Quinoa | Ethylparaben | 0.000000 |
| Protein Extract | | |
| Hydrolyzed Quinoa | DiSodium EDTA | 0.000000 |
| Protein Extract | | |
| Hydrolyzed Quinoa | Phenoxyethanol | 0.000000 |
| Protein Extract | | |
| Distilled Water | Water (Aqua, Eua) | 1.617300 |
| AMP-Ultra PC 2000 | Aminomethyl Propanol | 0.066690 |
| AMP-Ultra PC 2000 | Water (Aqua, Eau) | 0.003510 |
| Colorex HCB2 | HC Blue No. 2 | 1.140480 |
| Colorex DBL9 | 20721-50-0 | 0.123120 |
| Colorex HCY4 | HC Yellow No. 4 | 0.203040 |
| Colorex HCY2 | HC Yellow No. 2 | 0.123120 |
| Fusion Blue | Disperse Blue 377 | 0.570240 |
| Specially Denatured Alcohol | Alcohol Denat. | 10.786975 |
| (SDS) 40B-200 Proof | | |
| Specially Denatured Alcohol | t-Butyl Alcohol | 0.012960 |
| (SDS) 40B-200 Proof | | |
| Specially Denatured Alcohol | Denatonium Benzoate | 0.000065 |
| (SDS) 40B-200 Proof | | |
| Dimethyl Ether (DME) | Dimethyl Ether | 46.000000 |

### Example 5

The following example shows the maximum allowable percentage by weight, and ideal percentage by weight, of each chemical and compound comprising the ‘natural blonde’ colored embodiment of the invention.

| | | Breakdown |
|---|---|---|
| | | by |
| | INCI/Chemical | Percentage |
| | Names present in | wt. for each |
| Trade Name | the raw material | substance |
| Specially Denatured Alcohol | Alcohol Denat. | 48.4281 |
| (SDS) 40B-200 Proof | | |
| Specially Denatured Alcohol | t-Butyl Alcohol | 0.0582 |
| (SDS) 40B-200 Proof | | |
| Specially Denatured Alcohol | Denatonium Benzoate | 0.0003 |
| (SDS) 40B-200 Proof | | |
| Amphomer LV-71 Polymer | Octylacrylamide/ | 2.619000 |
| | Acrylates/ | |
| | Butylaminoethyl | |
| | Methacrylate | |
| | Copolymer | |
| Amphomer LV-71 Polymer | Water (Aqua, Eau) | 0.064800 |
| Amphomer LV-71 Polymer | PPG-12 Dimethicone | 0.002700 |
| Amphomer LV-71 Polymer | Benzoic Acid | 0.013500 |
| AMP-Ultra PC 2000 | Aminomethyl Propanol | 0.513000 |
| AMP-Ultra PC 2000 | Water (Aqua, Eau) | 0.027000 |
| Dow corning 556 | Phenyl Trimethicone | 0.054000 |
| Comsetic Grade Fluid | | |
| Citroflex 2 | Triethyl Citrate | 0.054000 |
| Volasil 995 | Cyclopentasiloxane | 0.212220 |
| Volasil 995 | Cyclohexasiloxane | 0.002592 |
| Volasil 995 | Cyclotetrasiloxane | 0.001188 |
| PC-AB Extract M6941-WS | Water (Aqua, Eau) | 0.002653 |
| PC-AB Extract M6941-WS | Glycerin | 0.002652 |
| PC-AB Extract M6941-WS | Citric Acid | 0.000014 |
| PC-AB Extract M6941-WS | Potassium Sorbate | 0.000014 |
| PC-AB Extract M6941-WS | Sodium Benzoate | 0.000014 |
| PC-AB Extract M6941-WS | Grapeseed Extract | 0.000009 |
| PC-AB Extract M6941-WS | Helianthus Annuus | 0.000009 |
| | (Sunflower) | |
| | Seed Extract | |
| PC-AB Extract M6941-WS | Persea Gratissima | 0.000009 |
| | (Avocado) Fruit | |
| | Extract | |
| PC-AB Extract M6941-WS | Calendula | 0.000009 |
| | Officinalis Flower | |
| | Extract | |
| PC-AB Extract M6941-WS | Aloe Barbadensis | 0.000009 |
| | Leaf Extract | |
| PC-AB Extract M6941-WS | Camellia Sinensis | 0.000009 |
| | Leaf Extract | |
| Fragrance: Enchanted | Fragrance (Parfum) | 0.144442 |
| Blossom #5198638 | | |
| Fragrance: Enchanted | Benzyl Benzoate | 0.003528 |
| Blossom #5198638 | | |
| Fragrance: Enchanted | Butylphenyl | 0.003402 |
| Blossom #5198638 | Methylpropional | |
| Fragrance: Enchanted | Hexyl Cinnamaldehyde | 0.001620 |
| Blossom #5198638 | | |
| Fragrance: Enchanted | Hydroxyisohexyl | 0.001620 |
| Blossom #5198638 | 3-Cyclohexene 3 | |
| | and carboxaldehyde | |
| Fragrance: Enchanted | Limonene | 0.003175 |
| Blossom #5198638 | | |
| Fragrance: Enchanted | Linalool | 0.004212 |
| Blossom #5198638 | | |
| Hydrolyzed Quinoa | Water (Aqua, Eua) | 0.000040 |
| Protein Extract | | |
| Hydrolyzed Quinoa | Hydrolyzed | 0.000014 |
| Protein Extract | Quinoa Protein | |
| Distilled Water | Water (Aqua, Eua) | 1.620000 |
| Fusion SP 8.0 Natural Blonde | HC Blue No. 2 | 0.040057 |
| Fusion SP 8.0 Natural Blonde | HC Orange No. 1 | 0.018457 |
| Fusion SP 8.0 Natural Blonde | HC Yellow No. 4 | 0.018457 |
| Fusion SP 8.0 Natural Blonde | Disperse Blue 377 | 0.010357 |
| Fusion SP 8.0 Natural Blonde | HC Yellow No. 2 | 0.010357 |
| Fusion SP 8.0 Natural Blonde | 4-Amino-Nitrophenol | 0.003240 |
| Fusion SP 8.0 Natural Blonde | 3-Nitro-p- | 0.003240 |
| | Hydroxy- | |
| | ethylaminophenol | |
| Fusion SP 8.0 Natural Blonde | HC Red No. 15 | 0.003240 |
| Fusion SP 8.0 Natural Blonde | Disperse Black 9 | 0.000594 |
| AMP-Ultra PC 2000 | Aminomethyl Propanol | 0.051300 |
| AMP-Ultra PC 2000 | Water (Aqua, Eau) | 0.002700 |
| Dimethyl Ether (DME) | Dimethyl Ether | 46.000000 |

It will be appreciated by persons skilled in the art that the present invention is not limited to what has been particularly shown and described hereinabove. A variety of modifications and variations are possible in light of the above teachings without departing from the scope and spirit of the invention.

What is claimed is:

1. An aerosolized, semi-permanent hair dye comprising:
a solvent;
a propellant;
a neutralizer;
a colorant treatment;
a colorant; and
a humectant

2. The invention of claim 1, wherein the colorant is a coal tar dye.

3. The invention of claim 2, further comprising a polymer.

4. The invention of claim 3, further comprising a plasticizer.

5. The invention of claim 4, further comprising Marketing Compounds.

6. The invention of claim 5, further comprising preservatives for Marketing Compounds.

7. The invention of claim 6, further comprising perfumes.

8. The invention of claim 7, further comprising hair conditioner.

9. The invention of claim 8, further comprising anti-dandruff compounds.

10. The invention of claim 9, further comprising a pH adjuster.

11. An aerosolized, semi-permanent hair dye comprising a solvent, a propellant, a neutralizer, a colorant treatment, a colorant, a humectant, a polymer, a plasticizer, Marketing Compounds, perfumes, a hair conditioner, an anti-dandruff compound, and a pH adjuster.
